(19) United States
(12) Patent Application Publication
(10) Pub. No.: US 2017/0172937 A1
(43) Pub. Date: Jun. 22, 2017

(54) SYSTEMS AND METHODS FOR PREPARING SOLID LIPID NANOPARTICLES

(71) Applicant: University of Mississippi, University, MS (US)

(72) Inventors: Michael Andrew Repka, Oxford, MS (US); Hemlata Gunga Patil, University, MS (US); Soumyajit Majumdar, Oxford, MS (US); Jun-Bom Park, University, MS (US); Vijay I. Kulkarni, University, MS (US)

(21) Appl. No.: 15/128,536

(22) PCT Filed: Mar. 24, 2015
(86) PCT No.: PCT/US2015/022213

§ 371 Date: Sep. 23, 2016

Related U.S. Application Data
(60) Provisional application No. 61/969,444, filed on Mar. 24, 2014.

(51) Int. Cl. A61K 9/51

(52) U.S. Cl. CPC A61K 9/5192; A61K 9/5123

(57) ABSTRACT

A continuous process for the manufacture of solid lipid nanoparticles includes preparing a pre-emulsion comprising a lipid and continuously passing the pre-emulsion through a high pressure homogenizer.

### CROSS-REFERENCE TO REPLATED APPLICATIONS

This application claims priority to U.S. provisional application No. 61/969,444, filed on Mar. 24, 2014, which is incorporated herein by reference in its entirety.

### FIELD OF THE DISCLOSURE

The present disclosure relates generally to preparing solid lipid nanoparticles in a continuous process.

### BACKGROUND

A solid lipid nanoparticle (SLN) system is a drug delivery system that has attracted increasing attention in recent years as a carrier system for cosmetic ingredients, nutraceuticals and pharmaceutical drugs. Solid lipid nanoparticle systems have been reported for controlled drug delivery, bioavailability enhancement by modification of dissolution rate and/or improvement of tissue distribution and targeting of drugs by using various application routes.

Solid lipid nanoparticles are mainly formed by non-solvent or solvent based techniques. The solvent based techniques utilize an organic solvent to dissolve the solid lipid and further evaporate it from the emulsion to obtain the solid lipid nanoparticles. The non-solvent techniques liquefy the solid lipid over its melting point and then convert it to a nanoemulsion through common techniques such as high pressure homogenization (HPH), high speed stirring or ultrasonication, and membrane emulsification. The nanoemulsion is then further cooled to obtain the solid lipid nanoparticles.

The process of solvent evaporation by precipitation in oil in water emulsions for formulations of solid lipid nanoparticles, however, has the disadvantage of the need for an organic solvent and the requirement of large amounts of surfactants. Further, although hot and cold high pressure homogenization have been explored for the feasibility in scaling-up the process, these methods for solid lipid nanoparticle preparation are multi-step batch processes (i.e., melting of the lipid, dispersion or dissolution of the drug in the melted lipid, preparation of an aqueous dispersion, size reduction, etc.).

As batch processes inherently have associated risks of batch to batch variation thus requiring careful and complex procedures and controls, continuous processes are typically preferred in the pharmaceutical industry over batch processes. Continuous processes can decrease the cost of production by needing less space, labor and resources, as well as by providing high efficacy and a better desired product quality as compared to a batch process. As such, it would be desirable to provide a continuous process for the formulation of solid lipid nanoparticles.

### SUMMARY

One embodiment is directed to a continuous process for the manufacture of solid lipid nanoparticles. The continuous process includes preparing a pre-emulsion comprising a lipid; and continuously passing the pre-emulsion through a high pressure homogenizer.

Another embodiment is directed to a continuous process for preparing solid lipid nanoparticles. The continuous process includes feeding a lipid composition through a hot-melt extruder to prepare a pre-emulsion; and continuously feeding the pre-emulsion through a high pressure homogenizer to form a solid lipid nanoparticle composition.

A further embodiment is directed to a system for preparing solid lipid nanoparticles. The system comprises a hot-melt extruder configured to prepare a lipid-containing pre-emulsion from a lipid feed; and a high pressure homogenizer coupled in series to the hot-melt extruder.

Another embodiment is directed to a process for preparing a pharmaceutical suitable for oral or parenteral administration. The process comprises feeding a lipid composition in combination with a drug through a hot melt extruder to prepare a pre-emulsion; and continuously feeding the pre-emulsion through a high pressure homogenizer coupled in series to the hot melt extruder to form a solid lipid nanoparticle.

### BRIEF DESCRIPTION OF THE DRAWINGS

FIG. 1 illustrates an exemplary system for preparing solid lipid nanoparticles.

FIG. 2 illustrates exemplary screws suitable for use in the system of FIG. 1.

FIG. 3 illustrates an effect of selected independent variables on entrapment efficiency.

FIG. 4 illustrates an effect of selected independent variables on particle size.

FIG. 5 illustrates an effect of selected independent variables on polydispersity index.

FIG. 6 illustrates an effect of selected independent variables on zeta potential.

FIG. 7 illustrates results of an in vitro drug release study.

FIG. 8 illustrates plasma drug concentration-time profiles.

FIGS. 9A-9C illustrate a particle size, polydispersity index, and zeta potential produced using an HME-HPH method and a conventional method.

Corresponding reference characters indicate corresponding parts throughout the drawings.

### DETAILED DESCRIPTION OF THE DRAWINGS

The processes and methods described herein utilize the processes of hot melt extrusion and high pressure homogenization to form solid lipid nanoparticles suitable for use in nutraceutical or pharmaceutical applications. The process is continuous and scalable and generally comprises mixing a lipid composition, optionally in combination with a drug, and a surfactant aqueous solution in an extruder barrel of a hot melt extruder at a temperature above the melting point of the lipid to form a pre-emulsion and further reducing the particle size of the pre-emulsion by high pressure homogenization. The hot melt extruder is connected in series to the high pressure homogenizer. Because the process described herein is a continuous process as opposed to a batch process, it is capable of providing better process controls and size reduction as compared to conventionally used batch processes. Moreover, the processes described herein may provide particles with an improved polydispersibility index and zeta potential over conventionally used batch processes.

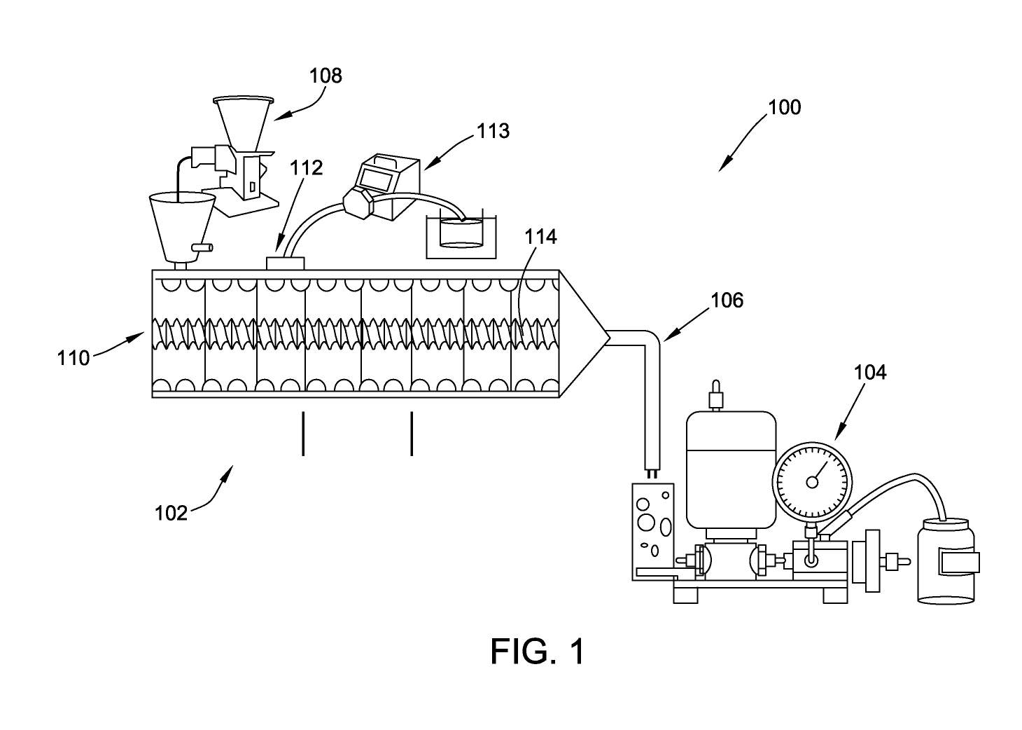

Referring now to the drawings, and in particular to FIG. 1, one embodiment of a system for preparing solid lipid nanoparticles is generally indicated at 100. In the embodiment shown in FIG. 1, system 100 includes a hot melt extruder 102 and a high pressure homogenizer 104 coupled in series by a connector 106. Hot melt extruder 102 may be any hot melt extruder known in the art. In one particular embodiment, hot melt extruder 102 is an 8-zone, co-rotating twin-screw extruder (11 mm Process 11, ThermoFisher Scientific, Karlsruhe, Germany).

A gravimetric feeder 108 is coupled to the extrusion barrel 110 of hot melt extruder 102 for feeding a lipid composition to hot melt extruder 102. An injection port 112 is coupled to extrusion barrel 110 at a zone downstream from gravimetric feeder 108. In one embodiment as shown in FIG. 1, gravimetric feeder 108 is coupled to extrusion barrel 110 at zone 1 and injection port 112, coupled to a peristaltic pump 113, is coupled to extrusion barrel 110 at zone 3. In an alternative embodiment, injection port 112, coupled to peristaltic pump 113, is coupled to extrusion barrel 110 at zone 4. One skilled in the art will understand that the location of gravimetric 108 and injection port 112 may be varied such that a suitable pre-emulsion (described below) may be obtained. Further, in some embodiments, system 100 may include multiple gravimetric feeders 108 each dispensing different components of a compositions into extrusion battle 110. Accordingly, feeding a lipid composition through a hot-melt extruder may refer to feeding one or more components of a lipid composition through a hot-melt extruder using one or more gravimetric feeders 108

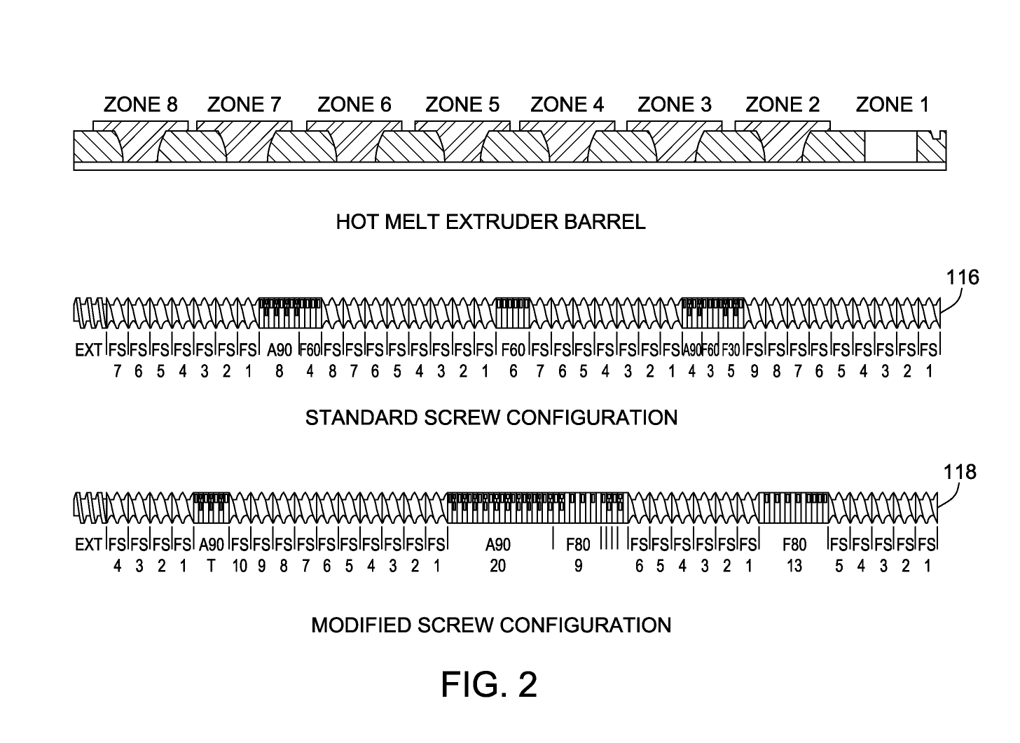

Extrusion barrel 110 includes at least one extrusion screw 114 positioned therein. Suitable extrusion screws are illustrated in FIG. 2 and are shown in a positional relationship with respect to zones 1-8 of one embodiment of extrusion barrel 110. In particular, extruder screw 116 is a standard screw configuration (Thermo Fisher Scientific, Walther, Mass.) and extruder screw 118 is a modified screw configuration comprising a mixing screw zone beginning from the point of contact between molten lipid and surfactant aqueous solution.

As noted above, hot melt extruder 102 is coupled to high pressure homogenizer 104 by connector 106. Connector 106 is coupled to a die (not shown) on hot melt extruder 102 and to a sample holder (not shown) of high pressure homogenizer 104. Connector 106 is an insulated tube so as to prevent heat loss of the pre-emulsion as the pre-emulsion passes from hot melt extruder 102 to high pressure homogenizer 104. Suitable materials for forming connector 106 include, but are not limited to, polyurethane or glass wool.

In operation, a lipid composition, such as glycerol behenate (Comptritol® 888 ATO, Precirol® ATO 5 (Gattefosse, France), and Imwitor® 900K (Cremer, Germany)), is fed into extruder barrel 110 of hot melt extruder 102 via gravimetric feeder 108. A surfactant aqueous solution, such as 1.5% w/w Tween 80 (Sigma Aldrich, USA), is further fed into extruder barrel 110 of hot melt extruder 102 via peristaltic pump 113 through injection port 112. Other suitable surfactant aqueous solutions include, but are not limited to, those having an HLB value of from about 12 to about 16 including Cremophore EL, and Vitamin E TPGS or an HLB value of greater than about 24 such as Pluronic F 68 (BASF, USA). In one embodiment, the lipid composition is fed through gravimetric feeder 108 at a first zone of extruder barrel 110 and the surfactant aqueous solution is fed through injection port 112 at either a third or fourth zone. It is understood by one skilled in the art, however, that the lipid composition and surfactant aqueous solution may be fed to extruder barrel 110 at any desired zone such that a suitable pre-emulsion is obtained.

A desirable pre-emulsion is obtained from hot melt extruder 102 by varying the formulation parameters, process parameters, and screw configurations of the hot melt extrusion process. For example, the formulation parameters such as the particular lipid composition and surfactant aqueous solution used in the process, are selected based on the desired pre-emulsion. In one particular embodiment, as noted above, the lipid composition may be glyceryl behenate (Compritol® ATO, Gattefosse, France) and the surfactant aqueous solution may be 1.5% w/w Tween 80 (Sigma Aldrich, USA).

Further, process parameters such as the rate at which the lipid composition and surfactant aqueous solution are added, the concentration of the lipid composition added, the speed of extrusion screw(s) 114, the zone of addition of each of the lipid composition and the surfactant aqueous composition, and the temperature of extruder barrel 110 along various zones may be varied to obtain the desired pre-emulsion. For example, in one particular embodiment, the lipid composition having a lipid concentration of about 6% w/w may be added to the first zone of extruder barrel 110, and the surfactant aqueous solution may be added to either of the third or fourth zones of extruder barrel 110. The rates of addition for each of the lipid composition and the surfactant aqueous solution vary based on the flow properties of the lipid composition and the desired output of the extrusion process. For example, in one embodiment, where a drug (e.g., fenofibrate) is added to extruder barrel 110 in combination with the lipid composition, for 100 ml/min extruder output and a ratio of fenofibrate:Compritol® of about 0.5:6, the solid feed rate (“SFR”) is about 100 rpm and the liquid feed rate (“LFR”) is about 47.75 ml/min. The lipid concentration may also be varied but should be done so with the understanding that increasing the lipid concentration may also require an increase in the rate at which the surfactant aqueous solution is added so as to avoid an insufficient surfactant concentration to lower the surface tension between the oil and water phase.

Extruder barrel 110 may include any number of extruder screws 114 in a variety of configurations. In one particular embodiment, extruder barrel 110 includes two extruder screws 114 in a modified configuration (such as extruder screw 116 shown in FIG. 2) within a co-rotating twin-screw extruder (11 mm, Process 11, ThermoFisher Scientific, Karlsruhe, Germany). In this embodiment, extruder screws 114 may have an operating screw speed of from about 150 RPM to about 250 RPM. Although the speed of extruder screws 114 may be varied, it should be understood that higher screw speeds may result in incomplete melting of the lipid composition at the point of addition of the surfactant aqueous solution and an insufficient contact time between the lipid composition and the surfactant aqueous solution. This may result in the formation of an inadequate pre-emulsion leading ultimately to a higher solid lipid nanoparticle size.

Further, as noted above, the zone at which each of the lipid composition and the surfactant aqueous solution may be added as well as the operating temperature of the various zones of extruder barrel 110 may vary the properties of the pre-emulsion. In one embodiment, the lipid composition is added at a first zone of extruder barrel 110 while the surfactant aqueous solution is added at either zone three or zone four of extruder barrel 110. One skilled in the art will understand that the zones of addition for each component may be varied to obtain a pre-emulsion with desired properties. Similarly, the operating temperature of each of these zones, and in particular the operating temperature for the mixing zones of extruder barrel 110, may also be varied to obtain an optimum pre-emulsion so long as the operating temperature is such that it is higher than that of the melting point of the lipid composition. The screw speed, which can be converted into residence time of the lipid (and optionally a drug) inside extruder barrel 110 can be modified to provide for a desired mixing zone within extruder barrel 110 based on the melting point of the lipid fed to extruder barrel 110.

The configuration of extruder screw(s) 114 may also vary the properties of the pre-emulsion. Specifically, certain geometries of extruder screw(s) 114 may allow for an increase in the radial mixing of material inside extruder barrel 110 by keeping the flow channels of the materials in contact with each other and by causing a higher shear rate inside extruder barrel 110. Increasing the amount of mixing elements can also increase the residence time. In one particular embodiment, by varying the above-referenced parameters, a pre-emulsion including about 6% w/w lipid, about 1.5% w/w surfactant, and about 92.5% w/w water may be formulated.

Once a suitable pre-emulsion is obtained, the pre-emulsion is passed through connector 106 and fed into high pressure homogenizer 104 through a sample holder (not shown in FIG. 1) for size reduction of the pre-emulsion. The high pressure homogenization process may be performed at varying process parameters, but in one embodiment, is performed at a temperature of about 75° C. and a pressure of from about 800 bar to about 1200 bar, more particularly about 800 bar to about 1000 bar, and more particularly about 1000 bar. In some embodiments, it is desirable for the temperature to be about 5° C. above the melting point of the lipid (and optionally the added drug) so that it will not solidify during passage. Once the homogenization process is complete, the resulting formulation is cooled at room temperature of about 25° C. to about 30° C. to obtain solid lipid nanoparticles.

Solid lipid nanoparticles formed from the process described above may have a mean particle size of from about 116 nm to about 310 nm, a zeta potential of from about −28 mV to about −35 mV, and a polydispersibility index of less than 0.5, including from about 0.25 to about 0.42. Mean particle size can be determined by Photon Correlation Spectroscopy (PCS) at 25° C. (Zetasizer-Nano-ZS, Malvern Instruments). Further, zeta potential, which reflects the electric charge on a particle surface and is useful in predicting the physical stability of colloidal systems, can also be determined using a Zetasizer-Nano-ZS (Malvern Instruments) at 25° C. The polydispersibility index is a dimensionless number calculated from a two parameter fit to the correlation data called a Cumulants analysis, known to those skilled in the art. The maximum value is limited to 1.0, indicating that the sample has a very broad size distribution and may contain large particles or aggregates that could be slowly sedimenting.

Suitable drugs for use in the above-described method include those belonging to the Biopharmaceutical Classification System (BCS) Class I, II, III, and IV. Particularly preferred drugs include those belonging to BCS class II and class IV (e.g., Glibenclamide, Bicalutamide, Ezetimibe, and Fenofibrate (class II) and Hydrochlorothiazide (class IV)) as these drugs are more lipid soluble. When a drug is used in the above-described method, the drug may be combined with a lipid and fed through a feeder into the extruder barrel wherein the drug further dissolves in the melted lipid.

The embodiments described herein utilize a system including a hot melt extruder connected in series to a high pressure homogenizer for preparing solid lipid nanoparticles. As hot melt extrusion technology is generally a fast, continuous manufacturing process, solid lipid nanoparticles are able to be formed in an efficient, continuous process thus minimizing batch to batch variations. That is, as the pre-emulsion for use in the high pressure homogenizer is able to be formed in a continuous process and coupled to the high pressure homogenizer in series, the quality of the pre-emulsion may be improved, thus improving the overall quality of the solid lipid nanoparticles while eliminating the need for costly and complex procedures and controls. In addition, compared to other known processes for producing solid lipid nanoparticles, such as preparing the pre-emulsion with ultra-turrex in a batch process, the above-described process produces solid lipid nanoparticles of a decreased particle size, decreased polydispersion index, and decreased zeta potential. Further, as continuous processes generally require fewer manual steps, the above-described process provides a faster, more efficient, and more cost effective method of solid lipid nanoparticle production with fewer variations and a better quality product.

### Example 1

The following Example illustrates a specific embodiment of a process for preparing solid lipid nanoparticles incorporating a drug. The Example is given solely for the purpose of illustration and is not to be construed as a limitation of the present disclosure, as many variations thereof are possible without departing from the spirit and scope of the disclosure.

In this Example, a hot melt extruder (co-rotating twin-screw extruder (11 mm Process 11, ThermoFisher Scientific, Karlsruhe, Germany)) including two rotating screws inside a stationary cylindrical barrel was configured with either a standard screw configuration or a modified screw configuration (both shown in FIG. 2). The extruder barrel consisted of eight zones. Zone one was configured for feeding a lipid and drug, and either zone three or zone four was configured for feeding a surfactant aqueous solution. The hot melt extruder was connected to a high pressure homogenizer via an insulated connector (similar to system 100 shown in FIG. 1).

A solid lipid (Compritol ATO 888; Imwitor 900K; Precirol or Dynasan) and Fenofibrate were fed into the extruder at zone 1 using a gravimetric feeder at a predetermined rate of about 50, about 75, or about 100 rpm with a ratio of Compritol ATO 888:Fenofibrate of about 6:0.25, about 6:0.5, or about 6:0.75. An aqueous surfactant solution comprising Tween 80 (Sigma Aldrich, USA) at about 1% w/w or Pluronic F 68 at about 1% w/w or Cremophore at about 0.25, 0.5, 1, 2, 3 or 5% w/w was added at either zone 3 or zone 4 at a temperature equivalent to the extrusion temperature through an injection port using a peristaltic pump at a predetermined rate in order to achieve the extruder output of about 50 ml/min., about 75 ml/min or about 100 ml/min. The melt-extrusion was performed by varying the formulation parameters and process parameters as shown in Table 1 below.

The hot pre-emulsion resulting from the melt extrusion was then passed through an insulated tube connecting the hot melt extruder die and the sample holder of the high pressure homogenizer (Avestin Emulsiflex C5, Canada). The high pressure homogenization was performed at a temperature of 75° C. and a pressure of 1000 bar to reduce the particle size of the pre-emulsion resulting from the melt-extrusion. The high pressure homogenization parameters were constant for each batch. The size reduced emulsion was cooled at room temperature (about 25° C. to about 30° C.) to obtain solid lipid nanoparticles.

A second batch of solid lipid nanoparticles were then prepared by a conventional batch method as described by Shengpeng Wang et al. using the same lipid and surfactant concentration as above. (Shengpeng et al., 2012. Emodin loaded solid lipid nanoparticles: Preparation, characterization and antitumor activity studies. International Journal of Pharmaceuticals. 430, 238-246.). The obtained pre-emulsion was then passed through a high pressure homogenizer.

The effect of process variables on particle size, polydispersibility index, and zeta potential of solid lipid nanoparticles produced by the continuous process described above are illustrated in Table 1 below. The solid lipid nanoparticles prepared by the above-described continuous process were further compared to the solid lipid nanoparticles prepared by the conventional batch process. The results of this comparison are also shown in Table 1 below.

The mean particle size was determined by Photon Correlation Spectroscopy (PCS) at 25° C. (Zetasizer-Nano-ZS, Malvern Instruments) directly after sampling. The dispersions were diluted with purified particle free water to an appropriate scattering intensity. The measurements were performed with eleven runs, each for 150 seconds, after an equilibration time of 120 seconds at 25° C. The results (the mean of the eleven measurements) are shown in Table 1 below as particle diameter (z-average) and the polydispersibility index (PDI), quantifying the size of the distribution of the nanoparticle population.

Zeta potential (ZP), which reflects the electric charge on the particle surface and is a useful parameter to predict the physical stability of colloidal systems, was also determined for each run. Zeta potential was determined using a Zetasizer-Nano-ZS (Malvern Instruments) at 25° C. For each measurement, the samples were diluted appropriately with ultrapurified water (pH of about 5.5).

TABLE 1

| | | | | | | | Extruder | | | Zeta |
|---|---|---|---|---|---|---|---|---|---|---|
| | | | | | | | output | Particle | | potential |
| Variables | Run | SC (%) | LC (%) | DC (%) | LIPID | SURFACTANT | (ml/min) | size (nm) | PDI | (mV) |
| LIPID | F1 | 1 | 6 | 0.5 | Compritol | Cremophore | 100 | 210.5 | 0.518 | −22.7 ± 3.1 |
| | | | | | 888 ATO | EL | | | | |
| | F2 | 1 | 6 | 0.5 | Imwitor | Cremophore | 10 | 259.2 | 0.806 | −21.9 ± 4.0 |
| | | | | | 900 K | EL | | | | |
| | F3 | 1 | 6 | 0.5 | Dynasan | Cremophore | 100 | 548 | 0.513 | −10.8 ± 4.2 |
| | | | | | 118 | EL | | | | |
| | F4 | 1 | 6 | 0.5 | Precirol | Cremophore | 100 | 749 | 0.721 | −7.9 ± 8.6 |
| | | | | | | EL | | | | |
| SURFACTANT | F5 | 1 | 6 | 0.5 | Compritol | Tween 80 | 100 | 642 | 0.662 | −9.2 ± 2.1 |
| | | | | | 888 ATO | | | | | |
| | F6 | 1 | 6 | 0.5 | Compritol | Pluronic F 68 | 100 | 631 | 0.6 | −17.2 ± 1.8 |
| | | | | | 888 ATO | | | | | |
| | F7 | 1 | 6 | 0.5 | Compritol | Cremophore | 100 | 210.5 | 0.518 | −26 ± 4.7 |
| | | | | | 888 ATO | EL | | | | |
| SURFACTANT | F8 | 0.25 | 6 | 0.5 | Compritol | Cremophore | 100 | 314.8 | 0.684 | −22.6 ± 1.4 |
| CONC | | | | | 888 ATO | EL | | | | |
| | F9 | 0.5 | 6 | 0.5 | Compritol | Cremophore | 100 | 241 | 0.485 | −34 ± 2.5 |
| | | | | | 888 ATO | EL | | | | |
| | F10 | 1 | 6 | 0.5 | Compritol | Cremophore | 100 | 210.5 | 0.518 | −32.6 ± 1.9 |
| | | | | | 888 ATO | EL | | | | |
| | F11 | 2 | 6 | 0.5 | Compritol | Cremophore | 100 | 248.5 | 0.464 | −12.6 ± 8.47 |
| | | | | | 888 ATO | EL | | | | |
| | F12 | 3 | 6 | 0.5 | Compritol | Cremophore | 100 | 178.4 | 0.386 | −33.2 ± 0.56 |
| | | | | | 888 ATO | EL | | | | |
| | F13 | 5 | 6 | 0.5 | Compritol | Cremophore | 100 | 116.1 | 0.423 | −34.1 ± 4.8 |
| | | | | | 888 ATO | EL | | | | |
| DRUG CONC | F14 | 1 | 6 | 0.25 | Compritol | Cremophore | 100 | 199.4 | 0.427 | −33.4 ± 1.9 |
| | | | | | 888 ATO | EL | | | | |
| | F15 | 1 | 6 | 0.5 | Compritol | Cremophore | 100 | 210.5 | 0.518 | −35 ± 4.2 |
| | | | | | 888 ATO | EL | | | | |
| | F16 | 1 | 6 | 0.75 | Compritol | Cremophore | 100 | 394.1 | 0.572 | −8.9 ± 4.6 |
| | | | | | 888 ATO | EL | | | | |
| | F17 | 1 | 6 | 1 | Compritol | Cremophore | 100 | 345 | 0.812 | −16.4 ± 1.8 |
| | | | | | 888 ATO | EL | | | | |
| | F18 | 1 | 6 | 2 | Compritol | Cremophore | 100 | 418.7 | 0.717 | −17.2 ± 5.08 |
| | | | | | 888 ATO | EL | | | | |
| LIPID CONC | F19 | 1 | 4 | 0.5 | Compritol | Cremophore | 100 | 193.6 | 0.508 | −22.7 ± 6.86 |
| | | | | | 888 ATO | EL | | | | |
| | F20 | 1 | 5 | 0.5 | Compritol | Cremophore | 100 | 218.4 | 0.614 | −27.1 ± 2.3 |
| | | | | | 888 ATO | EL | | | | |
| | F21 | 1 | 6 | 0.5 | Compritol | Cremophore | 100 | 210.5 | 0.518 | −28.4 ± 4.5 |
| | | | | | 888 ATO | EL | | | | |
| | F22 | 1 | 12 | 0.5 | Compritol | Cremophore | 100 | 784.1 | 0.712 | −21.2 ± 1.5 |
| | | | | | 888 ATO | EL | | | | |
| OTHER | F23 | 5 | 6 | 0.5 | Compritol | Cremophore | 100 | 116.1 | 0.423 | −34.1 ± 4.8 |
| | | | | | 888 ATO | EL | | | | |
| CONVENTIONAL | F23C | 5 | 6 | 0.5 | Compritol | Cremophore | — | 812.4 | 0.723 | −20.5 ± 1.7 |
| | | | | | 888 ATO | EL | | | | |
| | F24 | 5 | 6 | 0.5 | Compritol | Cremophore | 75 | 119.9 | 0.471 | −33 ± 1.9 |
| | | | | | 888 ATO | EL | | | | |
| | F25 | 5 | 6 | 0.5 | Compritol | Cremophore | 50 | 152.5 | 0.306 | −36.1 ± 3.5 |
| | | | | | 888 ATO | EL | | | | |
| | F26 | 5 | 6 | 0.25 | Compritol | Cremophore | 100 | 127.9 | 0.439 | −32 ± 2.4 |
| | | | | | 888 ATO | EL | | | | |
| | F27 | 5 | 6 | 0.25 | Compritol | Cremophore | 75 | 146.2 | 0.442 | −35 ± 1.6 |
| | | | | | 888 ATO | EL | | | | |
| | F28 | 5 | 6 | 0.25 | Compritol | Cremophore | 50 | 131.8 | 0.381 | −34.3 ± 3.1 |
| | | | | | 888 ATO | EL | | | | |
| | F29 | 5 | 6 | 0.75 | Compritol | Cremophore | 100 | 212.1 | 0.813 | −21.3 ± 4.5 |
| | | | | | 888 ATO | EL | | | | |
| | F30 | 5 | 6 | 0.75 | Compritol | Cremophore | 75 | 241.5 | 0.61 | −25.8 ± 4.2 |
| | | | | | 888 ATO | EL | | | | |
| | F31 | 5 | 6 | 0.75 | Compritol | Cremophore | 50 | 204.8 | 0.924 | −18.9 ± 2.7 |
| | | | | | 888 ATO | EL | | | | |
| | F32 | 0.25 | 6 | 0.5 | Imwitor | Cremophor | 100 | 413.3 | 0.548 | −19.3 ± 5.06 |
| | | | | | 900 K | EL | | | | |
| | F33 | 0.5 | 4 | 0.5 | Imwitor | Cremophor | 100 | 173.6 | 0.578 | −25.7 ± 6.86 |
| | | | | | 900 K | EL | | | | |
| | F34 | 0.5 | 6 | 0.5 | Imwitor | Cremophor | 100 | 188.2 | 0.476 | −20 ± 4.6 |
| | | | | | 900 K | EL | | | | |
| | F35 | 2 | 6 | 1 | Imwitor | Cremophor | 100 | 120 | 0.44 | −14.2 ± 4.56 |
| | | | | | 900 K | EL | | | | |
| | F36 | 2 | 6 | 1 | Imwitor | Cremophor | 100 | 128.1 | 0.464 | −10.3 ± 2.99 |
| | | | | | 900 K | EL | | | | |
| | F37 | 0.5 | 6 | 1 | Imwitor | Cremophor | 100 | 294 | 0.287 | −21.5 ± 5.61 |
| | | | | | 900 K | EL | | | | |
| | F38 | 0.5 | 6 | 2 | Imwitor | Cremophor | 100 | 381.7 | 1 | −19.6 ± 6 |
| | | | | | 900 K | EL | | | | |
| | F39 | 1 | 6 | 2 | Imwitor | Cremophor | 100 | 279.7 | 0.717 | −17.2 ± 5.08 |
| | | | | | 900 K | EL | | | | |
| | F40 | 5 | 6 | 2 | Imwitor | Cremophor | 100 | 148.6 | 0.485 | −10 ± 21.3 |
| | | | | | 900 K | EL | | | | |

Extruder Parameters: Screw Speed 250 RPM; Modified screw configuration; Zone 2 Temperature 150° C.; Zone 3 to Die Temperature 90° C.)
SC—surfactant concentration;
LC—lipid concentration;
DC—drug concentration

Table 2 below shows the particle size, PDI, and Zeta Potential for the production of a pre-emulsion run using hot melt extrusion (HME) (run F23) as compared to using a conventional process (run F23C).

TABLE 2

| | | Particle Size | | Zeta Potential |
|---|---|---|---|---|
| Run | Method | (nm) | PDI | (mV) |
| F23 | HME | 799.1 | 0.754 | −22.5 |
| F23C | Conventional | 1812.3 | 1.000 | −20.2 |

As shown in Table 2, particle size of the pre-emulsion was reduced from 1812.3 nm to 799.1 nm when the pre-emulsion was prepared by HME as compared to a conventional batch process as described in Shengpeng et al., 2012.

### Materials and Methods:

Preparation of Fenofibrate SLN:

Solubility Studies:

Solubility studies were performed to identify suitable lipids for the development of fenofibrate (FBT) solid lipid nanoparticles (SLN). The lipid used in the system should have high solubilization capacity for the drug, ensuring the solubilization of the drug in the resultant dispersion. The screening of lipids was performed by evaluating the solubility of FBT in different lipids such as Stearic acid, Compritol® 888 ATO, Precirol® ATO 5, Dynasan 114 and Imwitor® 900 K. Measured 10-25 mg of FBT was added into each vial containing 100 mg of selected excipient. Then, the mixture was heated at 90° C. in a water bath to facilitate the solubilization and the melt of drug and lipid were physically observed to confirm the presence or absence of insoluble drug crystals.

The assessment of solubility of drug in the lipid material is the first step in the selection of lipids for the formulation of SLN dispersions as solubility of the drug in the lipid is one of the most important factors for determining entrapment efficiency (EE) of the SLN. Four lipids with different physicochemical properties were selected and results from the solubility studies are shown in the following Table 3. In Table 3, ‘+’ indicates no drug crystal and ‘−’ indicates drug crystal seen at the end of test.

TABLE 3

mg Fenofibrate/100 mg solid lipid

| Lipid | 10 | 15 | 20 | 25 |
|---|---|---|---|---|
| Stearic acid | + | − | − | − |
| Compritol ATO | + | + | + | + |
| 888 | | | | |
| Precirol | + | + | + | − |
| Dynasan 114 | + | + | − | − |
| Imwitor 900K | + | − | − | − |

Among five lipids, FBT was not completely soluble in Stearic acid, Dynasan® 116 and Imwitor® 900K whereas no drug crystals were observed when FBT was heated together with Precirol® ATO 5 for all three concentrations tested (10-20 mg). Also when FBT was heated with Compritol® ATO 888, no drug crystals were observed for all four concentrations tested (10-25 mg). This study indicated that EE of FBT in Compritol® ATO 888 and Precirol® ATO 5 might be more than Dynasan® 116 and Imwitor® 900K. Thus Compritol® ATO 888 and Precirol® ATO 5 were selected for the preparation of SLN.

Design of Experiment:

A set of Plackett-Burmen (PB) screening design was adopted to study the effect of critical process parameters on FBT SLN performance and characteristics. PB design a commonly used design for experiments. They are the resolution of three designs so they can be used when only main effects of interest are to be investigated. PB designs involve a large number of variables and relatively fewer runs. A total of 12 experimental trials with PB design were constructed using design expert stat-ease software version 9. Multilinear regression analysis and one way ANOVA were performed to test the significance of the model and the factor coefficients. The experimental runs (formulations) were prepared in triplicate. The dependent variables were average particle size (Y1), polydispersibility index (Y2), zeta potential (Y3), entrapment efficiency (Y4). The linear equation of the model is as follows:

Y=b0+b1X1+b2X2+b3X3+b4X4+b5X5+ . . . +bnXn (Equation 1)

where Y is the response, b0 is the constant and b1, b2 . . . bn is the coefficient of factor X1, X2 . . . Xn (representing the effect of each factor ordered within −1, +1).

HPLC Analysis of Fenofibrate In Vitro:

A Waters HPLC-UV system (Waters Corp, Milford, Mass., USA) and UV detector set at wavelength of 286 nm. The separation of fenofibrate was performed on a Symmetry Shield RP 18 (5 μm) 4.6×250 mm column at 35° C., eluted with acetonitrile and phosphoric acid in water (pH=2.8) at a ratio of 85:15 (v/v). The mobile phase flow rate was maintained at 1.0 mL/min. Fenofibrate retention time was 7 min under these conditions. Injection volume was 20 μL. All of the HPLC data was analyzed using Empower V. software (Milford, Mass., USA). The calibration curve was linear with a correlation coefficient of 0.9998 over the range of 0.5-50 μg/ml. The within-day and between-day coefficients of variations did not exceed 3%. The limit of detection (LOD) value for fenofibrate was 15 ng/ml, and the limit of quantitation (LOQ) value was 50 ng/ml, respectively. The accuracy of the method was verified with recovery values of 98-102%.

### Characterization of FBT SLN:

Micromeritics Measurement:

The mean particle size and polydispersity index (PDI) of the developed SLN were determined by using Zetasizer Nano ZS (Malvern, USA). Dynamic Light Scattering technique was used to measure particle size. This technique measures the diffusion of particles moving under Brownian motion, and converts this to size and a size distribution using the Stokes-Einstein relationship. The mean value of three repeated measurements for each sample was reported as the final measurement. SLN sample was diluted with sufficient water and diluted sample were directly placed into cuvette and mean particle size and polydispersibility index were measured. Zeta potential measurement was carried out with the same instrument Zetasizer Nano ZS. All results are an average of three measurements, which are calculated based on an average of 10 runs. Zeta potential is defined as a measure of degree of repulsion between charged particles. These repulsive forces prevent the particle aggregation and are therefore an indicator of physical stability of the formulation. According to the literature dispersion with the zeta potential more than ±20 mV is physically stable (See, Shah R M, Malherbe F, Eldridge D, Palombo E A and Harding I H (2014) Physicochemical characterization of solid lipid nanoparticles (SLNs) prepared by a novel microemulsion technique. J Colloid Interface Sci 428:286-294). The parameters for zetasizer were set as scattering angle was 173°, refractive index was 1.33, viscosity was 0.89 cP and the temperature was 25° C.

Entrapment Efficiency Measurement:

Entrapment efficiency may be expressed as follows:

$$\frac{\begin{matrix}Total amount of FBT loaded - \\ Free FBT in supernatant\end{matrix}}{Total amount of FBT loaded} \times 100$$

For the determination of the percentage entrapment efficiency, the SLN were first separated from the aqueous suspension medium by ultrafiltration-centrifugation using centrifugal filters (Amicon Ultra—0.5 with 50 kDa cut-off, Millipore, USA) at 12000 RPM for 20 min at the room temperature. The percent entrapment efficiency was determined in triplicate indirectly by determining the amount of free FBT in the aqueous phase of the dispersion. The analysis of FBT was performed amount of free FBT in aqueous phase was measured by validated HPLC method at wavelength 288 nm. The separation of FBT was performed on a 250×4.6 mm BDS hypersil C18 column at 40° C., eluted with water and acetonitrile (25:75; pH adjusted to 2.6 with o-phosphoric acid) at a flow rate of 1.0 ml.min. The FBT entrapment efficiency (EE) of SLN was calculated from the amount of drug determined by the HPLC analysis.

Quantification of Drug Release In Vitro:

Dissolution tests for the crude FBT and the marketed micronized FBT formulation (200 mg) were performed with a dissolution apparatus using a paddle method. The accurately weighed amount of crude FBT (200 mg filled in gelatin capsules) and marketed FBT formulation were placed in 900 ml phosphate buffer pH 7.4 containing 0.3% sodium lauryl sulfate (SDS) at 37° C. and 75 rpm. An aliquot of 1.5 ml release media was withdrawn at intervals of 5, 10, 15, 30, 45, 60, 90, 120, 180, 240 and 300 min, and then replaced by 1.5 ml of fresh dissolution fluid. Each sample was passed through a 0.45 μm syringe filter and determined by HPLC. The drug release from SLN formulations was performed by using the dialysis bag technique. The dialysis bag method as previously used for studying drug release kinetics of nanoparticulate systems (See, Luo Y, Chen D, Ren L, Zhao X, Qin J. Solid lipid nanoparticles for enhancing vinpocetine's oral bioavailability. J Control Release. 2006; 114:53-59) with some modifications was used. Phosphate buffer pH 7.4 containing 0.3% SDS was used as the release medium. The dialysis bag retains nanoparticles and allows the free drug into the dissolution media with a cut-off of 10-14 kDa. The bags were soaked in double-distilled water for 12 hours (h) before use. Two milliliters of SLN dispersion was poured into the bag with the two ends fixed by clamps and immersed in 50 mL of pre-heated release medium in conical flask. The conical flasks were placed into a reciprocal shaking water batch manufactured by Precision (Cat. No 51221080) at 37° C. at a rate and 150 rpm. At fixed time intervals (Same time points which used for the dissolution of crude FBT and marketed formulation), the medium in the conical flask was removed and filtered for analysis and fresh dissolution medium was then added to maintain sink condition. The amount of drug in the filtrate was analyzed by HPLC method as described above. Measurements for all three crude FBT, marketed formulation and SLN formulation were performed in triplicate and averages are reported herein.

### Results and Discussion:

From preliminary studies, formulation parameters such as drug concentration (DC), surfactant concentration (SC), lipid concentration (LC), surfactant (ST), and lipid (LT) were found to have significant effects on characteristics of the SLN. Also, process parameters such as screw configuration, barrel temperature (BT), zone of liquid addition (ZA), and screw speed (SS) were found to have substantial effects. A modified screw configuration was selected to prepare all SLN formulations. To study the effect of formulation and process parameters, PB design approach was used. Experimental factors and their levels are given in Table 4 and the experimental design is shown in Table 5.

TABLE 4

| Factor | Factor Significance | Unit | Level (−1) | Level (+1) |
|---|---|---|---|---|
| X1 | Drug Concentration | % w/w | 0.5 | 1 |
| X2 | Lipid Concentration | % w/w | 6 | 8 |
| X3 | Surfactant | % w/w | 1.5 | 3 |
| | concentration | | | |
| X4 | Surfactant | — | Cremophore EL | Tween80 |
| X5 | Screw speed | RPM | 160 | 240 |
| X6 | Barrel Temperature | ° C. | 120 | 150 |
| X7 | Zone of Liquid | — | 3 | 4 |
| | addition | | | |
| X8 | Lipid | — | Compritol ® | Precirol ® |
| | | | ATO 888 | ATO 5 |

TABLE 5

| PB | X1 | X2 | X3 | X4 | X5 | X6 | X7 | X8 |
|---|---|---|---|---|---|---|---|---|
| 1 | 1 | 6 | 3 | Tween 80 | 160 | 150 | 4 | Precirol |
| 2 | 0.5 | 6 | 1.5 | Tween 80 | 160 | 150 | 4 | Compritol |
| 3 | 1 | 8 | 3 | Cremophore | 160 | 120 | 4 | Compritol |
| | | | | EL | | | | |
| 4 | 0.5 | 8 | 3 | Tween 80 | 160 | 120 | 3 | Precirol |
| 5 | 1 | 6 | 3 | Tween 80 | 240 | 120 | 3 | Compritol |
| 6 | 0.5 | 8 | 1.5 | Tween 80 | 240 | 120 | 4 | Precirol |
| 7 | 0.5 | 6 | 1.5 | Cremophore | 160 | 120 | 3 | Compritol |
| | | | | EL | | | | |
| 8 | 0.5 | 6 | 3 | Cremophore | 240 | 150 | 3 | Precirol |
| | | | | EL | | | | |
| 9 | 0.5 | 8 | 3 | Cremophore | 240 | 150 | 4 | Compritol |
| | | | | EL | | | | |
| 10 | 1 | 8 | 1.5 | Cremophore | 160 | 150 | 3 | Precirol |
| | | | | EL | | | | |
| 11 | 1 | 6 | 1.5 | Cremophore | 240 | 120 | 4 | Precirol |
| | | | | EL | | | | |
| 12 | 1 | 8 | 1.5 | Tween 80 | 240 | 150 | 3 | Compritol |

Experimental Design:

PB designs are screening designs that involve a large number of factors, which result in relatively fewer experiments. A total of 12 experimental trials involving 8 variables were performed and as shown in the following Table 6, the selected response variables exhibited a wide variation suggesting that the independent variables had a significant effect on the response parameters chosen. Observed and predicted values for all four responses are shown in Table 7.

TABLE 6

| | | | Polydispersibility | | | | Entrapment | |
|---|---|---|---|---|---|---|---|---|
| | Particle size (Y1) | | Index (Y2) | | Zeta Potential (Y3) | | Efficiency (Y4) | |
| Parameters | Coefficient | p-value | Coefficient | p-value | Coefficient | p-value | Coefficient | p-value |
| Constant | 354.58 | 0.007 | 0.46 | 0.042 | 31.25 | 0.047 | 61.0 | 0.0002 |
| b₀ | | | | | | | | |
| DC (A) | 43.25 | 0.056 | −0.01 | 0.829 | 0.02 | 0.981 | 0.30 | 0.230 |
| LC (B) | 48.25 | 0.039 | 0.08 | 0.019 | −2.35 | 0.045 | 3.67 | 0.0003 |
| SC (C) | −58.75 | 0.020 | −0.07 | 0.032 | 3.33 | 0.018 | −0.08 | 0.705 |
| ST (D) | 48.08 | 0.040 | 0.06 | 0.056 | −0.70 | 0.396 | −7.64 | <0.0001 |
| SS (E) | −2.75 | 0.9079 | 3.752E−017 | 1.0000 | 1.70 | 0.0957 | 2.79 | 0.0008 |
| BT (F) | −62.75 | 0.0155 | −0.01 | 0.7400 | 0.77 | 0.3572 | −0.61 | 0.0552 |
| ZA (G) | 8.58 | 0.7209 | 0.16 | 0.5263 | 0.21 | 0.7889 | −1.18 | 0.0095 |
| LT (H) | 97.58 | 0.0025 | 0.06 | 0.0690 | −4.02 | 0.0108 | −5.64 | <0.0001 |

TABLE 7

| PB | Actual | Predicted | Actual | Predicted | Actual | Predicted | Actual | Predicted |
|---|---|---|---|---|---|---|---|---|
| Run | Y1 | Y1 | Y2 | Y2 | Y3 | Y3 | Y4 | Y4 |
| 1 | 397.00 | 330.50 | 0.385 | 0.44 | −33.24 | −32.92 | 39.13 | 39.75 |
| 2 | 209.00 | 252.83 | 0.507 | 0.47 | −31.12 | −31.08 | 51.17 | 50.25 |
| 3 | 289.00 | 261.17 | 0.463 | 0.37 | −35.02 | −35.08 | 75.01 | 74.75 |
| 4 | 462.00 | 552.50 | 0.576 | 0.57 | −24.42 | −24.08 | 50.28 | 49.75 |
| 5 | 349.00 | 260.83 | 0.257 | 0.30 | −39.34 | −38.92 | 60.34 | 59.75 |
| 6 | 674.00 | 670.00 | 0.765 | 0.74 | −22.38 | −21.92 | 53.18 | 53.25 |
| 7 | 245.00 | 282.17 | 0.296 | 0.32 | −34.04 | −33.92 | 68.84 | 68.75 |
| 8 | 153.00 | 234.33 | 0.376 | 0.29 | −36.16 | −36.08 | 62.49 | 61.75 |
| 9 | 125.00 | 135.67 | 0.284 | 0.37 | −39.28 | −38.92 | 78.46 | 78.25 |
| 10 | 542.00 | 448.33 | 0.534 | 0.59 | −19.49 | −18.92 | 65.08 | 64.75 |
| 11 | 485.00 | 477.33 | 0.454 | 0.46 | −27.71 | −27.08 | 62.19 | 61.75 |
| 12 | 325.00 | 349.33 | 0.625 | 0.59 | −32.82 | −32.08 | 66.24 | 66.25 |

The responses can be mathematically expressed as follows:

Y1=+354.58+43.25*X1+48.25*X2−58.75*X3+48.08*X4−2.75*X5−62.75*X6+8.58*X7+97.58*X8 (Equation 2)

Y2=+0.46−0.0072X1+0.081*X2−0.070*X3+0.059*X4−0.00833*X6+0.016*X7+0.055*X8 (Equation 3)

Y3=+31.25+0.018*X1−2.35*X2+3.32*X3−0.70*X4+1.70*X5+0.77*X6+0.21*X7−4.02*X8 (Equation 4)

Y4=+61.03+0.30*A+3.67*X2−0.083*X3−7.64*X4+2.78*E−0.61*X6−1.18*X7−5.64*X8 (Equation 5)

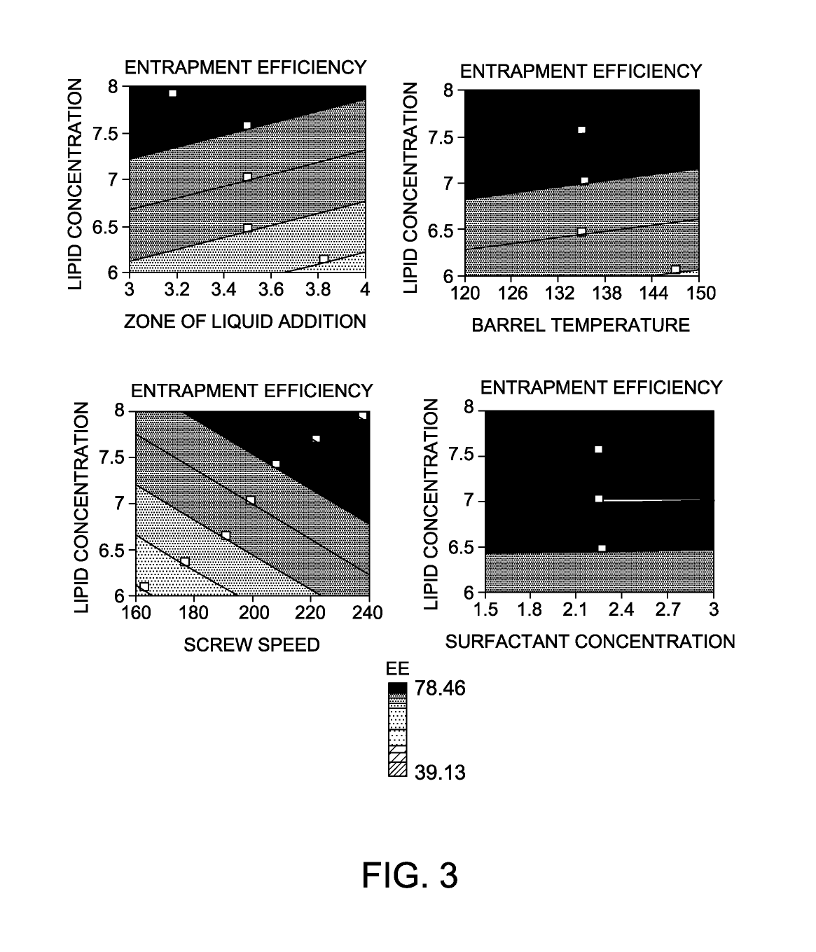

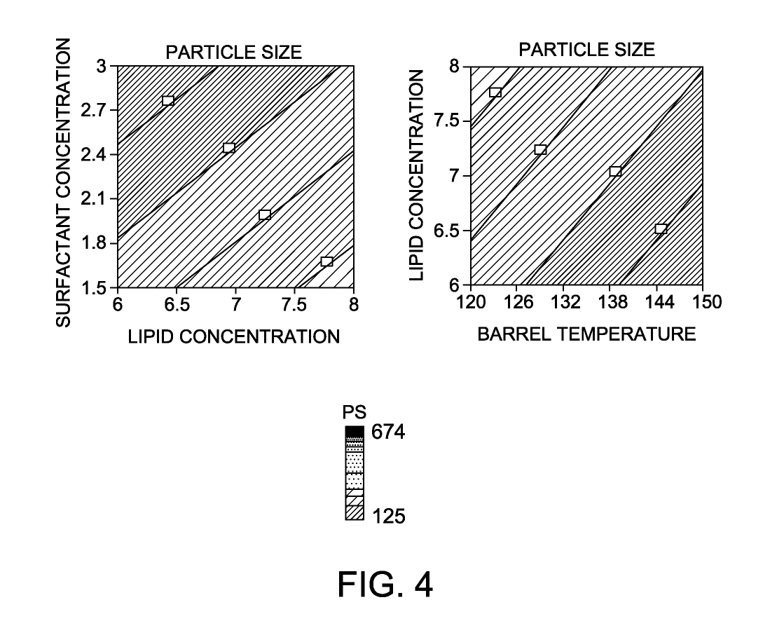

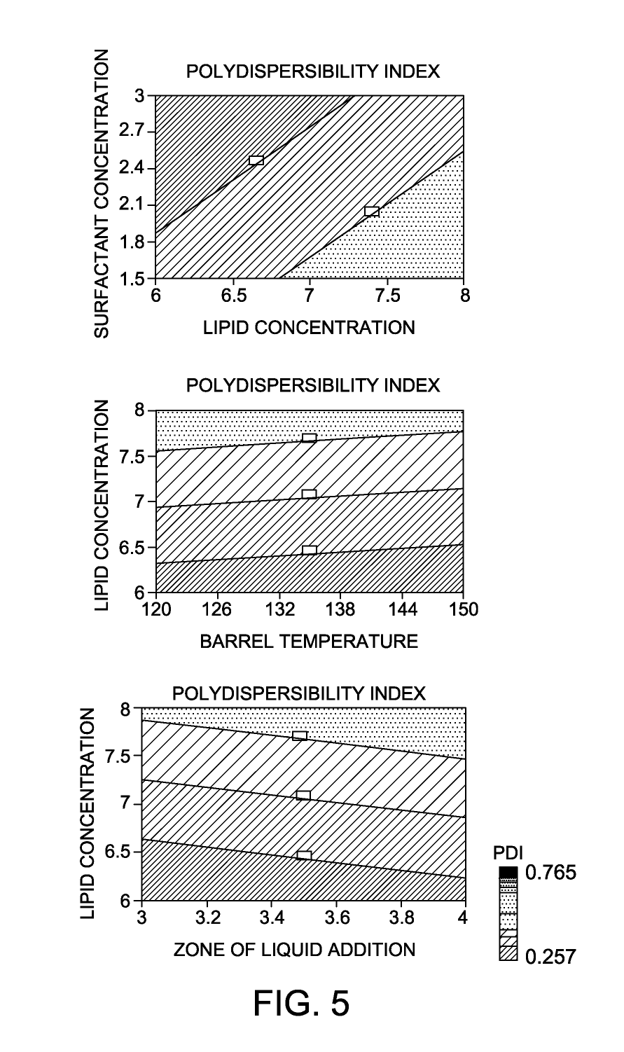

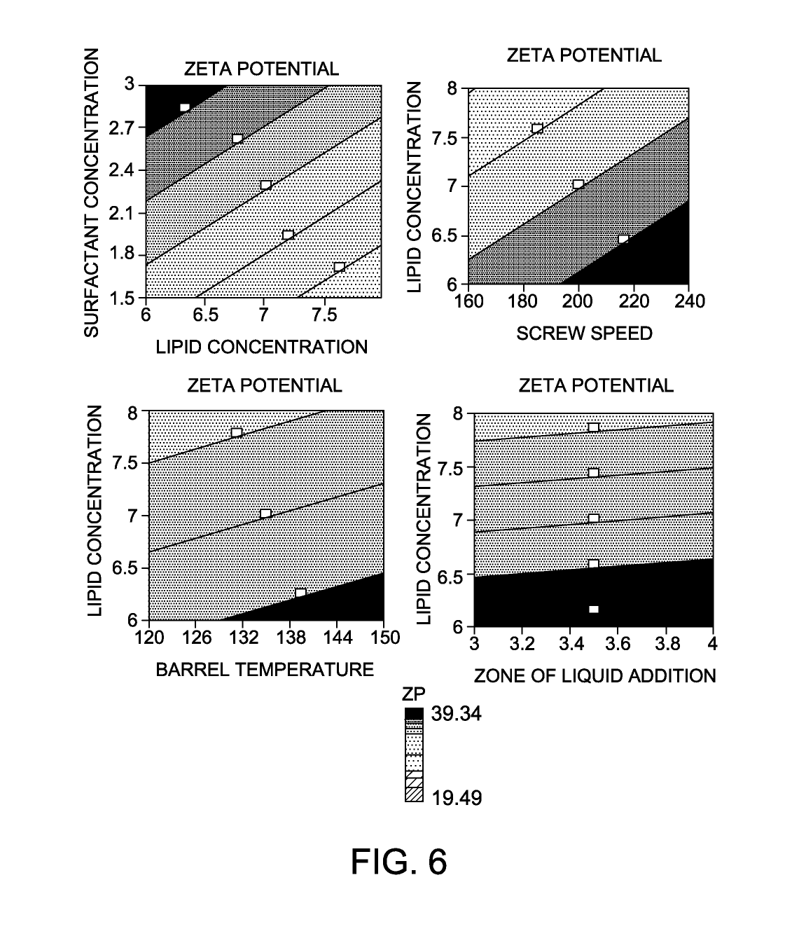

The coefficients in Equations 2-5 represent the respective quantitative effect of the independent variables (X1, X2, X3, X4, X5, X6, X7 and X8) on the response variables (Y1, Y2, Y3 and Y4). The effect of selected independent variables on entrapment efficiency (EE), particle size (PS), polydispersity index (PDI), and zeta potential (ZP) is graphically shown in FIGS. 3, 4, 5, and 6, respectively. The effects of various parameters will now be described in detail.

Effect of Drug Concentration:

The drug concentration was varied at two levels as follows, 0.5 and 1 g. As shown in Equations 2, 4 and 5, drug concentration had positive coefficients for PS, ZP and EE but the p value for the drug concentration was found to be more than 0.05 indicating that the drug concentration had insignificant effects on all four responses: PS, PDI, ZP as well as EE. This could be due to the high solubility of FBT in both lipids Compritol® ATO 888 and Precirol® ATO 5, which might result in less variations in the viscosity of the oily phase and therefore there is lower shear generated inside the extruder barrel. Increasing drug concentration results in the increase in drug-to-lipid ratio, which usually decreases EE. Surprisingly, the results showed that increasing drug concentration did not decrease the EE of the drug. This may be due to the range of drug concentration we have used in the formulation. The concentration range from 0.5-1% w/w may be solubilizing the drug completely into the lipid and therefore there is no decrease in EE, as was observed. It may possible that drug concentration is increased beyond 1% w/w, decrease in EE of the drug might be observed.

Effect of Surfactant Concentration:

Concentration of surfactant demonstrated a significant influence on particle size and PDI. As shown in Equation 2, the negative value of the coefficient for the surfactant concentration indicates particle size and PDI was decreased with an increase in SC. This might be due to the production and stabilization of smaller lipid droplets at higher SC as enough surfactant was present to reduce interfacial tension between two immiscible phases and stabilize the nano-droplets. SC had a significant effect (P<0.05) on ZP. Equation 5 gives the positive coefficient for SC indicating an increase in SC increases ZP. Generally SC exhibits a huge influence on entrapment efficiency. As described in Rahman Z, Zidan A S, Habib M J, Khan M A, “Understanding the quality of protein loaded PLGA nanoparticles variability by Plackett-Burman design” Int J Pharm. 2010; 389(1-2): 186-194, a higher amount of surfactant increases the solubility of the drug in the external phase and might be increasing the partitioning of drug from the internal phase to the external phase. Therefore, generally entrapment efficiency increases with an increase in SC as the presence of sufficient SC, which helps drug to remain within the lipid particles and/or the surface of the particles. Surprisingly, the SC had a negative coefficient but p-value was more than 0.05 indicating SC demonstrated an insignificant effect on EE (as shown in Equation 5).

Effect of Lipid Concentration:

Lipid concentration did show a significant effect on all four responses: PS, PDI, ZP and EE. Particle size significantly increased with increasing lipid concentration. Presence of high lipid concentration results increase in the viscosity of the drug-lipid melt. This viscosity increase might cause less homogenization during the initial phase of emulsification and produce larger particles and larger PDI. The negative coefficient of lipid concentration from Equation 4 indicates an increase in LC decreases the ZP of the SLN formulation. This may be due to the negative charge of the lipid. As the amount of negatively charged lipid increases in the formulation, the ZP of formulation decreases. As expected, EE significantly increased with increasing LC. This might be due to availability of a higher amount of lipid to encapsulate more drug, which led to higher EE.

Effect of Different Surfactant:

Choice of surfactant showed a significant effect on particle size of SLN. Positive coefficient for ST indicates changing ST from Cremophore EL to Tween 80 increases the particle size. Cremophore EL produces SLN with smaller particle size than Tween 80. Similar observations are reported by other researchers (See, Das S, Ng W K, Kanaujia P, Kim S, Tan R B. Formulation design, preparation and physicochemical characterizations of solid lipid nanoparticles containing a hydrophobic drug: effects of process variables. Colloids Surf B Biointerfaces. 2011; 88:483-489). This might be due to the difference in HLB value of Cremophore EL and Tween 80. HLB value of 1 and 2 for Compritol® ATO 888 and Precirol® ATO 5, respectively, are closer to the HLB of 12-14 for Cremophore than HLB of 15 for Tween 80. ST showed a relatively large effect on EE. As mentioned previously, surfactant increases the solubility of the drug in the external phase and might be increasing the partitioning of drug from the internal phase to the external phase. As shown in Equation 5, the negative coefficient for ST indicates that changing ST from Cremophore EL to Tween 80 decreases EE. This may be again due to the high HLB value of Tween 80. EE was significantly low when Tween 80 was used.

Effect of Screw Speed:

Screw speed produced an insignificant effect (p>0.05) on PS, PDI and ZP. On the other hand it exhibited a significant effect on EE. The positive coefficient for screw speed indicates increase in speed results in the increase in EE. High screw speed generates higher shear inside the barrel, which might result in more homogenization causing more interaction of drug, lipid and surfactant resulting in the formation of a homogeneous emulsion. This may explain the increase in EE.

Effect of Barrel Temperature:

Barrel temperature essentially only affected particle size of the SLN. The negative coefficient of barrel temperature for particle size demonstrated that increasing barrel temperature reduces the particle size of SLN. This might be because with high barrel temperature, it is possible that the lipids and drug are completely melted resulting in a low viscosity melt without any solid particles. Barrel temperature (zone 2) had insignificant effects on PDI, ZP and EE of SLN.

Effect of Zone Liquid Addition:

Zone of liquid addition showed an insignificant effect on PS, PDI and ZP. Surprisingly, however, ZA demonstrated a very significant effect (P=0.0095) on entrapment efficiency. Negative coefficient for ZA indicates changing zone of liquid addition from zone 3 to zone 4 decreases EE. This is may be due to the difference in temperature of molten mass at zone 3 and zone 4. The temperature of the molten mass is higher in zone 3 than zone 4 which could affect mixing of the molten mass with the surfactant aqueous solution and droplet formation. Also the other reason to decrease EE by changing the zone of liquid addition could be the length of mixing elements inside the barrel from zone 4 to die is less as compared to the mixing elements from zone 3 to die (see FIG. 2). If surfactant solution is added at zone 3 then the molten mass of lipid-drug and surfactant solution are exposed to a larger mixing zone, which generates high shear and may enhance entrapment of drug inside the molten lipid. Addition of surfactant solution at zone 4 provides less exposure to the molten lipid-drug mass for mixing with the surfactant solution. Therefore, addition of liquid at zone 4 results in a decrease in EE (39.8%).

Effect of Different Lipid:

Different lipids exhibited a positive impact on particle size and Compritol® ATO 888 produced smaller SLN when used as the lipid. This may be due to the variations in melt viscosity of Compritol® ATO 888 and Precirol® ATO 5. The positive coefficient for LT for PDI indicates low PDI obtained with Compritol® ATO 888 than Precirol® ATO 5 but p-value for LT was found to be more than 0.05 indicating an insignificant effect on PDI. EE was found higher in the case of Compritol® ATO 888 as compared to Precirol® ATO 5. These observations can be explained by the solubility study that demonstrated the higher solubilization capacity of Compritol® ATO 888 for FBT.

Quantification of Drug Release In Vitro:

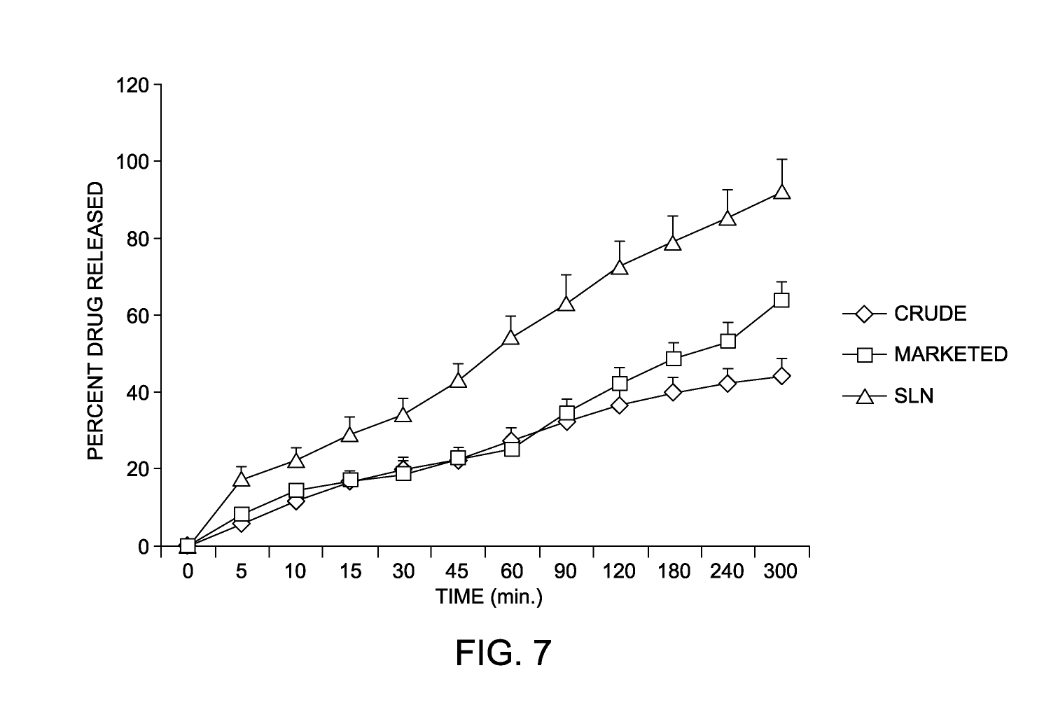

As aqueous solubility of FBT is very low, 0.3% SDS was added to the release media to maintain the sink condition. In vitro release of FBT from the optimized formulation (PB 9) was compared with the release of pure drug and the marketed micronized FBT formulation for a period of 5 h. The amount of FBT released from the SLN dispersion was determined by an in vitro dialysis bag technique. The dialysis bag retained the SLN particles; drug released from SLN and diffused through the dialysis membrane into the release media. In vitro drug release studies (shown in FIG. 7) showed approximately 94% drug was released at the end of 5 h for FBT SLN formulation (PB 9) and 62% for the commercially available micronized FBT formulation at the end of 5 h, whereas 41% drug was released from pure FBT at the end of 5 h. The increase of the dissolution rate of SLN FBT could be mainly attributed to the obvious reduction of the particle size (from a few microns for the crude FBT and marketed micronized formulation to a few nanometers (125 nm) for SLN). According to Nernst-Noyes-Whitney equation, which described the dissolution rate of drug in a diffusion-controlled process, an increase in the surface area could result in an increase in dissolution rate.

In Vivo Pharmacokinetic Study:

All animal care and experimental studies were approved by the Institutional Animal Care and Use Committee (IACUC) with protocol no. 14-013. Jugular vein cannulated male Wistar rats (body weight 250±10 g, Harlan laboratories, Indiana, USA) were housed in cages for a minimum of at least three days prior to the beginning of the study and had free access to food and water. Rats were randomly divided into three groups of six animals each. The rats were fasted for 12 h prior to experiments and were given access to food after the experiments. The oral dose of FBT was 12.5 mg/animal, thus 0.5 ml formulation was administered to each rat. The crude FBT drug was suspended in 0.1% SLS aqueous solution and the commercial micronized FBT formulation was diluted 1:10 with saline prior to administration by gavage. The SLN formulation was administered undiluted. Serial blood samples (200 μl) was taken from the cannulated jugular vein, pre-dose and at time points of, 0.5, 1, 2, 4, 6, 8, 10, 24 h post-dosing. The whole blood was collected into heparin coated tubes and centrifuged at 4° C. at 12,000 rpm for 5 min to obtain plasma. The plasma samples were kept frozen at −80° C. until analysis.

Plasma Processing and HPLC Analysis:

Fenofibric acid was determined by an HPLC-UV method as describe above. The sample preparation of the plasma samples was based on the procedure described in Hanafy A, Spahn-Langguth H, Vergnault G, Grenier P, Tubic Grozdanis M, et al., “Pharmacokinetic evaluation of oral fenofibrate nanosuspensions and SLN in comparison to conventional suspensions of micronized drug”, Adv Drug Deliv Rev. 2007; 59:419-426, with modifications. An aliquot of 100 μl plasma and 400 μl methanol was transferred to Eppendorf tubes and vortexed for 1 min, followed by the centrifugation at 12,000 rpm for 10 min at 4° C. An aliquot of 20 μl supernatant was injected into the HPLC system and the fenofibric acid was detected. The standard calibration curve was prepared in plasma similarly as described above. The standard curve was obtained, with a correlation coefficient of 0.9998 over the concentration range from 0.11 μg/ml to 123.68 μg/ml. The recovery by the described procedure was more than 92% in the investigated concentration range.

Data and Statistical Analysis:

The pharmacokinetic parameters were calculated based on a non-compartmental model. The area under the concentration-time curve from time zero to time t (AUC0-t) was calculated using trapezoidal method. Peak concentration (Cmax) and time of peak concentration (Tmax) were obtained directly from the concentration-time profiles. Differences between batches were analyzed by one-way analysis of variance (ANOVA) followed by Tukey test. P<0.05 was considered statistically significant. All values were reported as mean of four findings.

Conclusion:

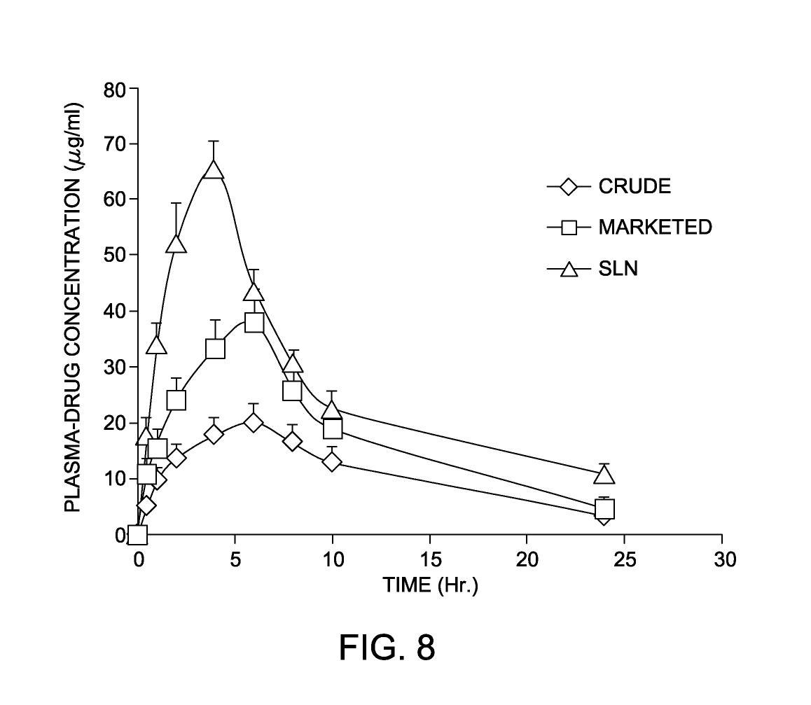

In order to determine whether the increase in the dissolution rate helps to improve the oral bioavailability, an in vivo experiment was conducted in the fasted state. The pro-drug FBT, which contains an ester group, undergoes rapid hydrolysis to produce fenofibric acid by intestinal, plasma and tissue esterase, following oral administration. Thus, in the present study the pharmacokinetic analysis of FBT is based on the plasma concentration of fenofibric acid. The plasma drug concentration-time profiles of FBT after oral administration of various formulations to male Wistar rats is shown in FIG. 8. The related pharmacokinetic parameters are summarized in Table 8.

TABLE 8

| | | | Micronized |
|---|---|---|---|
| | | | marketed FBT |
| Parameters | SLN Formulation | Crude FBT | formulation |
| Tmax (h) | 4.42 ± 0.4*^{#} | 6.9 ± 0.54 | 6.5 ± 0.4 |
| Cmax | 65.25 ± 7.2*^{#} | 20.0 ± 3.5 | 38.05 ± 5.8 |
| (μg/ml) | | | |
| AUC₀₋₂₄ | 648.23 ± 85.4* | 266.17 ± 40.9 | 430.97 ± 70.0 |
| (h · μg/ml) | | | |
| AUMC_{0-24 h} | 5342.17 ± 527.2*^{#} | 2306.74 ± 221.1 | 3509.26 ± 324.1 |
| (μg-h₂/ml) | | | |
| MRT₀₋₂₄ (h) | 8.24 ± 0.5 | 8.66 ± 1.4 | 8.14 ± 0.7 |

*P < 0.05 compared with crude FBT
^{#}P < 0.05 compared with Marketed FBT

At all-time points, the fenofibric acid plasma concentration was significantly higher (P<0.05) for the rats treated with fenofibrate-SLN than the marketed and crude fenofibrate. The Cmax value of fenofibrate SLN (65.3±7.2 μg/ml) was higher than the marketed (38.05±5.8 μg/ml) and crude fenofibrate (20.0±3.5 μg/ml). Twenty-four hours after oral administration, the fenofibrate-SLN plasma concentration was 10 μg/ml which was higher as compared to the marketed formulation (4 μg/ml) and crude fenofibrate (3 μg/ml). From these results, one can conclude that fenofibrate absorption was enhanced significantly by employing the SLN formulations compared to the marketed and crude fenofibrate (31-32). The most important advantage of SLN formulations over the crude fenofibrate is the lipid protection of drug from chemical as well as enzymatic degradation, thus further delaying the in vivo metabolism by oxidative and conjugative pathways. An increase in the rate of drug absorption from the SLN has been demonstrated compared to the both marketed micronized formulation and crude drug suspension. Reduction in particles size from the micro- to nanometer range increases the surface area and thus increases the dissolution velocity according to the equation of Noyes-Whitney.

Comparison Between the SLN Prepared by Conventional Method and HME-HPH Method:

From PB design, three optimized formulations PB 7, PB 8 and PB 9 were selected and prepared by a conventional method. Briefly, in conventional method lipid and drug heated up to temperature 10-15° C. above the melting point of lipid and drug. Surfactants were dissolved in beaker with water, and then added into melted drug and lipid drop by drop over 70-800 C water bath. The obtained pre-emulsion was passed through a high pressure homogenizer. These three formulations were compared for characteristics such as PS PDI and ZP when prepared by conventional and by HME-HPM techniques.

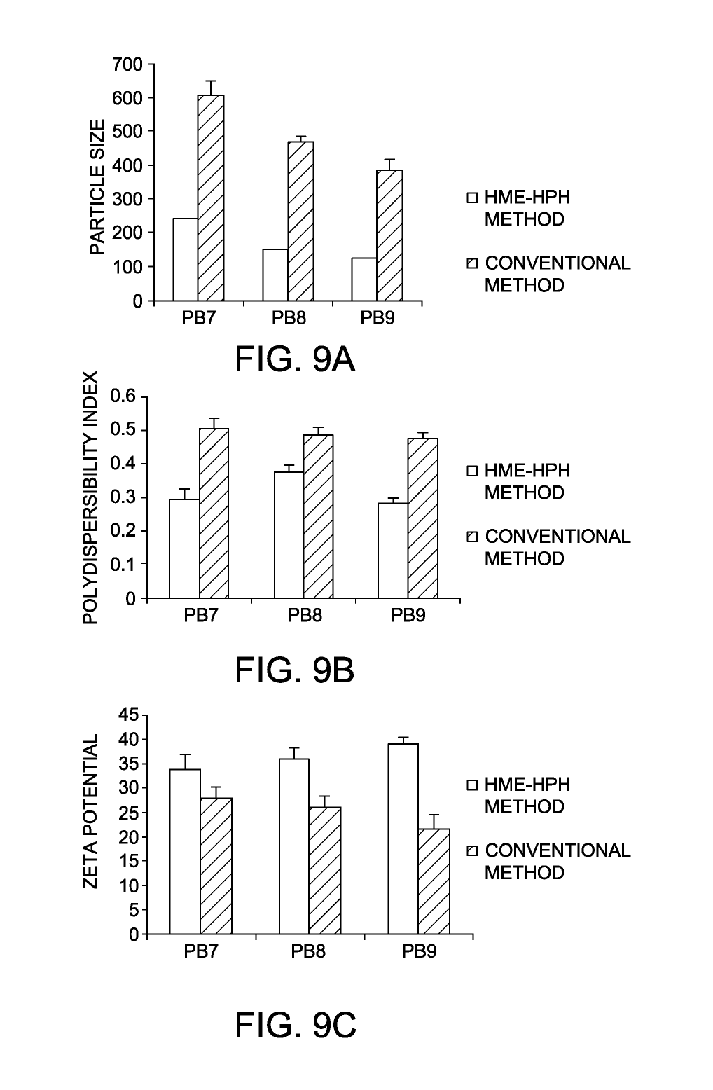

The particle size, PDI and ZP of the SLN produced using the HME-HPH method and conventional method are shown in supplementary FIGS. 9A-9C. The pre-emulsion prepared by HME and the conventional method compared with each other are emphasized as this is the major contributing difference in these two methods. The quality of the pre-emulsion affects the quality of the final product attributes to a large extent and it is desirable to obtain droplets in the size range within a few micrometers. Pre-emulsions prepared by both the HME-HPH method and the conventional method were characterized for PS, PDI and ZP. Particle size of the pre-emulsion prepared by the conventional method was 1643 nm whereas the PS was 653 nm when prepared by the HME-HPH method (See Table 9). It is apparent that if the initial pre-emulsion particle size is significantly higher that it will take more cycles and pressure in the HPH to reduce the particle size to 150-200 nm. However, the particle size of the pre-emulsion prepared by the HME-HPH method is itself in nanometers; less downstream processing in the HPH is required. With this HME:HPH method high pressure-induced drug degradation that could be caused by HPH can be prevented because of the requirement of fewer number of cycles as well as lower pressure as compared to the conventional SLN preparation method.

TABLE 9

| | | Particle size | | Zeta Potential |
|---|---|---|---|---|
| No. | Method | (nm) | PDI | (mV) |
| 1 | HME | 653 | 0.63 | −24.1 |
| 2 | Conventional method | 1643 | 1.21 | −8.2 |

Stability Study:

The stability of the developed SLN formulation was conducted for 6 months. The optimized formulation (PB 9 design) was selected for the stability study. Briefly, samples were stored in the sealed amber colored glass vials at 4° C. and at 25° C. After 1, 3 and 6 months, the samples were characterized with respect to particle size, ZP, PDI, and EE.

Stability results are shown in Table 10. Particle size, PDI and zeta potential studies revealed no statistically significant change (P value>0.05). Particle size increased after 6 months storage at 25° C. A slight reduction in entrapment efficiency of the FBT SLN was observed after 6 months storage at 25° C. and at 4° C. However the changes were not statistically significant. Thus it can be concluded that FBT SLN have good physical stability in terms of particle size, PDI, ZP and EE when stored at 4° C. and 25° C. for the 6 month study period.

TABLE 10

| | | | | | Zeta Potential | | Entrapment Efficiency | |
|---|---|---|---|---|---|---|---|---|
| | Particle Size (nm) | | PDI | | (mV) | | (%) | |
| Storage | 4° C. | 25° C. | 4° C. | 25° C. | 4° C. | 25° C. | 4° C. | 25° C. |
| Fresh | 125 ± 0.16 | | 0.284 ± 0.032 | | 39.28 ± 1.21 | | 78.46 ± 1.42 | |
| 1 | 124.2 ± 0.4 | 128 ± 0.1 | 0.286 ± 0.04 | 0.284 ± 0.04 | 39.04 ± 1.7 | 38.23 ± 1.4 | 78.13 ± 1.3 | 77.92 ± 1.4 |
| month | | | | | | | | |
| 3 | 127 ± 0.3 | 133 ± 0.5 | 0.292 ± 0.1 | 0.293 ± 0.02 | 37.74 ± 1.5 | 36.12 ± 1.2 | 77.48 ± 1.9 | 75.14 ± 1.2 |
| month | | | | | | | | |
| 6 | 132 ± 0.3 | 139 ± 1.4 | 0.301 ± 0.1 | 0.302 ± 0.02 | 37.22 ± 1.3 | 35.12 ± 2.4 | 76.23 ± 2.1 | 73.47 ± 1.5 |
| month | | | | | | | | |

As demonstrated by the results described above, solid lipid nanoparticles may be successfully prepared by the above-described continuous process including hot melt extrusion and high pressure homogenization. The quality of the pre-emulsion affects the quality of the final solid lipid nanoparticles. As shown in Table 1 above, better results were achieved with the above-described continuous process as compared to a conventional batch process using ultra-turrex in combination with high pressure homogenization. In particular, the solid lipid nanoparticles produced by the above-described continuous process as compared to conventional batch processes have a smaller mean particle size, a lower polydispersibility index, and a higher zeta-potential. Further, as the above-described process is a continuous process, there are less variations in the formulations resulting in a higher quality product than in formulations produced with batch processes. The above-described process involves fewer manual steps, therefore it is a fast and cost effective process for producing solid lipid nanoparticles.

When introducing elements of the present invention or preferred embodiments thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions and methods without departing from the scope of the invention, it is intended that all matter contained in the above description and shown in the accompanying drawings shall be interpreted as illustrative and not in a limiting sense.

1. A continuous process for the manufacture of solid lipid nanoparticles, the process comprising:
preparing a pre-emulsion comprising a lipid; and
continuously passing the pre-emulsion through a high pressure homogenizer.

2. The process of claim 1 wherein preparing a pre-emulsion comprises passing a lipid composition through a hot melt extruder.

3. The process of claim 2 wherein preparing the pre-emulsion further comprises adding a surfactant to the hot melt extruder, wherein the surfactant has a hydrophile-lipophile balance value of from about 12 to about 16.

4. The process of claim 2 wherein the hot melt extruder comprises two rotating screws of a modified screw configuration.

5. The process of claim 2 wherein the hot melt extruder comprises eight zones.

6. The process of claim 5 wherein the lipid composition is added to the hot melt extruder at zone one and the surfactant is added to the extruder at one of zone three or zone four.

7. The process of claim 3 wherein the lipid composition and surfactant are each added at a rate such that the pre-emulsion comprises about 6% weight/weight lipid, about 1.5% weight/weight surfactant, and about 92.5% weight/weight water.

8. The process of claim 2 wherein the pre-emulsion is passed in sequence from the hot melt extruder to the high pressure homogenizer via an insulated connector.

9. The process of claim 1 wherein the pre-emulsion is passed through the high pressure homogenizer at a pressure of about 1000 bar and a temperature of about 75° C.

10. The process of claim 1 wherein the process produces solid lipid nanoparticles having a mean particle size of from about 116 nm to about 310 nm, a zeta potential of from about −28 mV to about −35 mV, and a polydispersibility index of less than 0.5.

11. A continuous process for preparing solid lipid nanoparticles, the continuous process comprising:
feeding a lipid composition through a hot-melt extruder to prepare a pre-emulsion; and
continuously feeding the pre-emulsion through a high pressure homogenizer to form a solid lipid nanoparticle composition.

12. The process of claim 11 further comprising cooling the solid lipid nanoparticle composition.

13. The process of claim 11 wherein preparing the pre-emulsion further comprises adding a surfactant to the hot melt extruder, wherein the surfactant has a hydrophile-lipophile balance value of from about 12 to about 16.

14. The process of claim 13 wherein the hot melt extruder comprises eight zones.

15. The process of claim 14 wherein the lipid composition is added to the hot melt extruder at zone one and the surfactant is added to the extruder at one of zone three or zone four.

16. The process of claim 15 wherein the lipid composition and surfactant are each added at a rate such that the pre-emulsion comprises about 6% weight/weight lipid, about 1.5% weight/weight surfactant, and about 92.5% weight/weight water.

17. The process of claim 11 wherein the hot melt extruder comprises two rotating screws of a modified screw configuration.

18. The process of claim 11 wherein the pre-emulsion is passed in sequence from the hot melt extruder to the high pressure homogenizer via an insulated connector.

19. The process of claim 11 wherein the pre-emulsion is passed through the high pressure homogenizer at a pressure of about 1000 bar and a temperature of about 75° C.

20. The process of claim 11 wherein the process produces solid lipid nanoparticles having a mean particle size of from about 116 nm to about 310 nm, a zeta potential of from about −28 mV to about −35 mV, and a polydispersibility index of less than about 0.5.

21. (canceled)

22. (canceled)
